SEQUENCE LISTING (8 sequences)

SEQ ID NO: 1 - DNA, 112 bp, Artificial Sequence
  Description of Artificial Sequencesynthesized
cgatttaggtgacactatagaactcacctatctchhhhhhhhhhhhhhhhhhhhhhhhhhhhhhhhhhhhhhhhhhhhhhhhhhhhhhhhhaatggaagacgccaaaaacat

SEQ ID NO: 2 - DNA, 20 bp, Artificial Sequence
  Description of Artificial Sequencesynthesized
gtcagaccccgtagaaaaga

SEQ ID NO: 3 - DNA, 20 bp, Artificial Sequence
  Description of Artificial Sequencesynthesized
tctgagaactcacctatctc

SEQ ID NO: 4 - DNA, 20 bp, Artificial Sequence
  Description of Artificial Sequencesynthesized
tatgcagttgctctccagcg

SEQ ID NO: 5 - DNA, 20 bp, Artificial Sequence
  Description of Artificial Sequencesynthesized
ggagagcaactgcataaggc

SEQ ID NO: 6 - DNA, 20 bp, Artificial Sequence
  Description of Artificial Sequencesynthesized
agcgtcagaccccgtagaaa

SEQ ID NO: 7 - DNA, 20 bp, Artificial Sequence
  Description of Artificial Sequencesynthesized
cttatgcagttgctctccag

SEQ ID NO: 8 - DNA, 59 bp, Artificial Sequence
  Description of Artificial Sequencesynthesized
gaacucaccuaucucauacaacuuucaacuuccuauuucuacacaaaacauuucccuac

(19) United States
(12) Patent Application Publication
(10) Pub. No.: US 2006/0029999 A1
(43) Pub. Date: Feb. 9, 2006

(54) Nucleic acid sequences having an activity of regulating translation efficiency and utilization thereof

(75) Inventors: Tatsuya Sawasaki, Matsuyama-shi (JP); Yaeta Endo, Matsuyama-shi (JP); Nami Kamura, Matsuyama-shi (JP)

Correspondence Address: KILYK & BOWERSOX, P.L.L.C., 400 HOLIDAY COURT, SUITE 102, WARRENTON, VA, 20186, US

(73) Assignee: CellFree Sciences Co., Ltd.

(21) Appl. No.: 11/053,594

(22) Filed: Feb. 8, 2005

(30) Foreign Application Priority Data
Aug. 4, 2004 (JP) JP2004-227866

(51) Int. Cl. C12N 15/867; C07H 21/04; C12P 21/06; C07K 14/47

(52) U.S. Cl. 435/69.1; 435/456; 435/325; 530/350; 536/23.5

(57) ABSTRACT

The present invention provides a polynucleotide comprising a nucleic acid sequence having an activity of regulating the translation efficiency of a template in a cell-free protein synthesis system and also provides a method for utilizing the same, etc. Protein synthesis is carried out by a translation template containing a polynucleotide comprising a nucleic acid sequence which is to be an object to be selected, a polyribosome fraction is prepared from the reaction solution and a nucleic acid sequence bonding to ribosome is analyzed whereupon a selection is done.

### TECHNICAL FIELD

The present invention relates to a nucleic acid sequence having an activity of regulating the translation efficiency of a template in a protein synthesis system, a polynucleotide comprising the nucleic acid sequence and utilization of the polynucleotide, etc.

### BACKGROUND OF THE INVENTION

Intracellular protein synthesis reactions proceed in such steps that, firstly, genetic information is transcribed to mRNA from DNA having the information and the information of mRNA is translated on ribosome whereupon protein is synthesized. As to a method for conducting such an intracellular protein synthesis in vitro, there has been briskly conducted the development of a method where, for example, a component containing ribosome, etc. which is a protein translation apparatus equipped in the cells is extracted from living body and is conducted in vitro by addition of a transcription or translation template followed by adding nucleic acids, amino acids, various ions, buffer and other effective factors which are to be substrates to the extract (hereinafter, such a series of operations may be referred to as “cell-free protein synthesis system”) (Patent Documents 1 to 5, etc.).

Cell-free protein synthesis systems have capacities comparable to those of living cells in terms of rate of peptide synthesis and correctness of translation reaction. In addition, these have advantages that complicated chemical reaction steps and troublesome cell culture steps are not necessary. Moreover, in recent years, there have been conducted developments in order to further enhance the translation efficiency that a group of nucleases, translation-inhibiting protein factors, proteases, etc. contaminated into extracts of tissues or cells used for the conventional cell-free protein synthesis system are inactivated (Patent Document 6), the contamination as such is prevented (Patent Document 7), etc.

On the other hand, utilization for improvement in efficiency for protein synthesis having a sequence for improving the translation efficiency itself has been known as well. Such a translation promotion sequence include the 5′ cap structure (Non-Patent Document 1), Kozak sequence (Non-Patent Document 2) and the like in eukaryotes. The Shine-Dargarno sequence and the like are known in prokaryotes. Moreover, it has been found that a translation promoting activity in a 5′-untranslated leader sequence of RNA virus as well (Non-Patent Document 8) and a method where protein synthesis is efficiently carried out using those sequences has been developed (Non-Patent Document 9). However, due the reason that those translation promotion sequences have specificity to RNA polymerase which is subjected to transcription, it is hardly able to be concluded that they are suitable for utilizing in protein synthesis.

On the other hand, there has been developed a method where, from a polynucleotide group having artificially random sequence, that which shows an activity for regulating the translation efficiency has been developed (Non-Patent Document 10).

[Patent Document 1] Japanese Patent Laid-Open No. 06/98,790

[Patent Document 2] Japanese Patent Laid-Open No. 06/225,783

[Patent Document 3] Japanese Patent Laid-Open No. 07/194

[Patent Document 4] Japanese Patent Laid-Open No. 09/291

[Patent Document 5] Japanese Patent Laid-Open No. 07/147,992

[Patent Document 6] Japanese Patent Laid-Open No. 2000/236,896

[Patent Document 7] Japanese Patent Laid-Open No. 2000/236,896

[Patent Document 8] Japanese Patent No. 2,814,433

[Patent Document 9] Japanese Patent Laid-Open No. 10/146,197

[Patent Document 10] International Laid Open WO 03/056,009

[Non-Patent Document] Shatkin, Cell, 9, 645-(1976)

[Non-Patent Document] Kozak, Nucleic Acid. Res., 12, 857-(1984)

### SUMMARY OF THE INVENTION

Problems to be solved by the present invention is to provide a novel polynucleotide having an activity of regulating the translation efficiency of translation template in a cell-free protein synthesis system prepared using a method where, from a polynucleotide group containing artificially random sequence, that having an activity of regulating the translation efficiency is selected; a translation template containing the polynucleotide; a method for synthesis of protein by a protein synthesis system using the translation template; etc.

The present inventors have conducted intensive studies for solving the above-mentioned problems and, as a result, they have found a novel polynucleotide which raises the translation efficiency when a cell-free protein synthesis is conducted by a wheat germ extract using a translation template for synthesis of luciferase protein containing polynucleotide of 20 to 300 mer having a random sequence, then a polyribosome fraction is recovered from the reaction solution by means of a sucrose density gradient centrifugal method, a sequence analysis of the above random sequence contained in a translation template in the fraction is conducted and protein synthesis is conducted using a translation template containing a polynucleotide comprising the sequence. The present invention is achieved on the basis of such a finding.

In accordance with the present invention, there are provided a nucleic acid sequence which is an artificial sequence being unavailable in nature and having an activity of regulating the translation efficiency and a polynucleotide comprising the sequence. When the polynucleotide is used, it is now possible to conduct a protein synthesis with a very high efficiency in a protein synthesis system.

Thus, the present invention provides the followings.

1. A polynucleotide having a translation enhancement activity containing the following sequence:

1) nucleic acid comprising a sequence represented by SEQ ID NO: 8 of the Sequence Listing and/or a complementary chain thereof,

2) nucleic acid coding for a sequence comprising a sequence where one to several nucleotide(s) in a sequence represented by SEQ ID NO: 8 of the Sequence Listing is/are substituted, deleted, inserted or added and/or a complementary chain thereof,

3) nucleic acid having at least 80% of homology to a sequence represented by SEQ ID NO: 8 of the Sequence Listing and/or a complementary chain thereof.

2. A polynucleotide having a translation enhancement activity containing a sequence which is hybridized to the sequence mentioned in the above 1 under a stringent condition or a polynucleotide comprising a complementary sequence thereof.

3. The polynucleotide according to the above 1 or 2, wherein the activity of regulating the translation efficiency is identical with or higher than the activity of a 5′-untranslated leader sequence of RNA virus.

4. A translation template containing the polynucleotide mentioned in any of the above 1 to 3.

5. A method for the protein synthesis wherein the translation template mentioned in the above 4 is used.

6. A protein which is prepared by the method for the protein synthesis mentioned in the above 5.

7. A vector containing the polynucleotide mentioned in any of the above 1 to 3.

### BRIEF DESCRIPTION OF DRAWINGS

FIG. 1 illustrates the outline of a system which selects a sequence having an activity of promoting translation.

FIG. 2 shows a pattern where polysome in a translation reaction solution is fractionated by means of a sucrose density gradient centrifugation.

FIG. 3 is a graph where using fluorescence of GFP as an index, an Ω sequence is compared with an activity for promoting translation of a novel sequence obtained from the polysome sequence.

FIG. 4 is a drawing of SDS-PAGE where using synthesized amount of GFP as an index, an Ω sequence is compared with an activity for promoting translation of a novel sequence obtained from the polysome sequence.

### DETAILED DESCRIPTION OF THE INVENTION

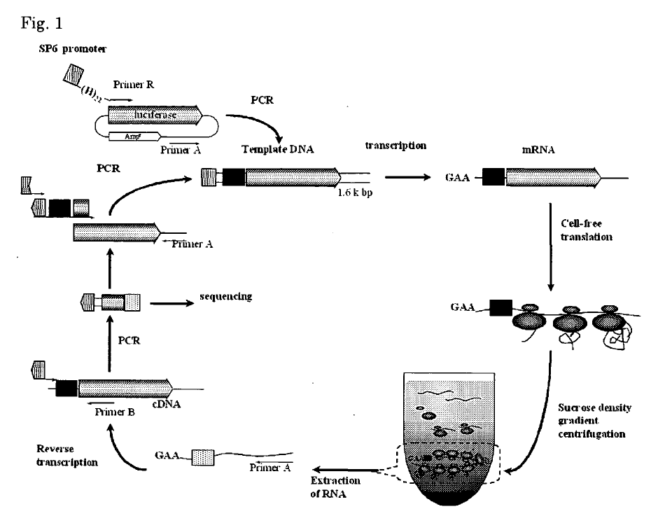

The present invention relates to a novel polynucleotide which is selected using a method mentioned in Patent Document 10 from a polynucleotide group containing an artificially random sequence and has an activity of regulating the translation efficiency of template in a cell-free protein synthesis system and also to utilization of the polynucleotide. FIG. 1 shows an outline of the process mentioned in Patent Document 10. The present invention will now be illustrated in more detail as hereunder.

(1) Preparation of a Translation Template Containing Polynucleotide Comprising Free Nucleic Acid Sequence Which is Used as an Object for Selection

With regard to a nucleic acid sequence to be selected, although anything may be used so far as it is a sequence which is able to have an activity for regulating the translation efficiency, a sequence which is a random sequence having a length of 3 to 200 mer and has no start codon is used preferably.

With regard to a polynucleotide group having the sequence as such (hereinafter, that may be referred to as “candidate polynucleotide”), there may be listed a process where, in the conventional synthetic method for oligonucleotide, the column used therefor is made for containing a mixture of nucleic acids having four kinds of different bases to conduct a chemical synthesis in the case of a random sequence. Here, in order to give a random sequence containing no start codon, a process for synthesis by a mixture of nucleic acids containing no one or more kind(s) of A, T and G among the above four kinds of bases or by a mixture of nucleic acids where any one kind is changed to inosine or the like may be preferably used. In case a random sequence is used, it is preferred that a sequence of the polynucleotide is analyzed or, in order to amplify by a polymerase chain reaction (PCR), a common sequence is added to its 5′-terminal. With regard to the common sequence, there is no particular limitation so far as it has no start codon and has a sequence where primer of PCR is able to be annealed. With regard to its chain length, a length of 3 to 50 mer is preferred.

With regard to a candidate polynucleotide, it is bonded in a manner of being sandwiched between an appropriate promoter sequence and a polynucleotide having start codon and coding for polypeptide (that may be referred to as “coding polynucleotide” in the present specification) whereupon a translation template is prepared. With regard to a polypeptide for which a coding polynucleotide is coded, anything may be used so far as it is able to be synthesized in a protein synthesis system. However, since synthetic amount of the polypeptide is an index for changes in translation efficiency by the candidate polynucleotide, that which issues a signal being easily observed such as fluorescence is preferred and that where the signal amount and the protein amount are correlated is preferred. Examples of such a polypeptide are luciferase and GFP.

It is preferred that the coding polynucleotide comprises not only a coding region of the above-mentioned polypeptide but also a 3′-untranslated region containing a transcription termination region, etc. With regard to the 3′-untranslated region, about 0.1 to 3.0 k bases which is downstream from stop codon is preferably used. It is not always necessary that the 3′-untranslated region as such is that of the coding polynucleotide per se. With regard to promoter, that which is specific to RNA polymerase used for the transcription thereafter may be used. Specific examples thereof are SP6 promoter and T7 promoter.

With regard to the promoter and also to a bonding method for a coding polynucleotide with a candidate polynucleotide, it is possible to use a common method which has been known per se. To be more specific, for bonding of promoter with a candidate polynucleotide, a method where promoter sequence of 5′-side is continuously synthesized in chemical synthesis of a candidate polynucleotide is used. With regard to a method for bonding with a coding polynucleotide, there may be used, for example, a method where, in a PCR using a coding polynucleotide as a template, a sense primer which is a candidate polynucleotide synthesized by bonding a promoter sequence is bonded with an antisense primer which is a polynucleotide comprising a 3′-terminal sequence of 3′-untranslated region.

It is also preferred that a sequence having an activity for controlling the transcription and the translation efficiency is further inserted. With regard to the sequence and the inserting position therefor, it is preferred in the case of eukaryotes that a 5′ cap structure (Shatkin, Cell, 9, 645-(1976)) is inserted to a 5′-terminal of a translation template and a Kozak sequence (Kozak, Nucleic Acid. Res., 12, 857-(1984)) is inserted between the coding polynucleotide and the candidate polynucleotide of the present invention while, in the case of prokaryotes, it is preferred a Shine-Dargarno sequence is inserted between the coding polynucleotide and the candidate polynucleotide of the present invention.

(2) Reaction for Synthesis of Protein using a Translation Template

A translation template containing a candidate polynucleotide of the present invention is translated if necessary and then subjected to a protein synthesis reaction. With regard to the protein synthesis system, anything may be used so far as it has an ability of being able to produce a protein by translating the translation template and, to be more specific, living cells and cell-free protein synthesis system may be exemplified. With regard to a cell-free protein synthesis system, known ones such as Escherichia coli, plant seed germ and extract of cells such as reticulocytes of rabbits may be used. With regard to the above, that which is available in the market may be used or that may be prepared according to a method known per se such as, in the case of an extract of E. coli, it is able to be prepared by a method mentioned in Pratt J. M., et al., Transcription and Translation, Hames, 179-209, B. D. & Higgins, S. J., eds.), IRL Press, Oxford (1984).

With regard to a commercially available cell-free protein synthesis system or cell extract, examples of that derived from E. coli are E. coli S30 extract system (manufactured by Promega) and RTS 500 Rapid Translation System (manufactured by Roche), examples of that derived from reticulocytes of rabbit are Rabbit Reticulocyte Lysate System (manufactured by Promega), etc. and examples of that derived from wheat germ are Proteios™ (manufactured by Toyobo), etc. Among them, it is preferred to use a system of extract of plant seed germ. With regard to plant seeds, those of plants of Gramineae such as wheat, barley, rice and corn and of spinach are preferred. In a cell-free synthesis system, a protein synthesis system having a high ability of polyribosome formation is preferred and, therefore, it is advantageous to use an extract of wheat germ.

With regard to a method for the preparation of a wheat germ extract, methods mentioned, for example, in Johnston, F. B., et al., Nature, 179, 160-161 (1957), Erickson, A. H., et al., (1996), Meth. In Enzyymol., 96, 38-50, etc. may be used. It is also preferred to conduct a treatment for removal of translation inhibitory factor contained in the extract such as endosperm containing tritin, thionine, nuclease, etc. (Japanese Patent Laid-Open No. 2000/236,896, etc.) or a treatment for the suppression of a translation inhibitory factor (Japanese Patent Laid-Open No. 07/203,984). The cell extract prepared as such is able to be used for a protein synthesis system by the same method as the conventional one.

A composition for a synthesis reaction solution used for the protein synthesis system of the present invention includes the above-mentioned cell extract, a translation translation template containing candidate polynucleotide, substrate amino acid, energy source, various ions, buffer, ATP regeneration system, nuclease inhibitor, tRNA, reducing agent, polyethylene glycol, 3′,5′-cAMP, antibacterial agent, etc. When DNA is used as a translation translation template, it is possible to further contain substrates for RNA synthesis, RNA polymerase, etc. They are appropriately selected depending upon the aimed protein and to the type of the protein synthesis system used and then prepared.

The amino acid used as a substrate is twenty kinds of amino acids constituting a protein. Examples of energy sources include ATP and GTP. Examples of various ions include acetate such as potassium acetate, magnesium acetate and ammonium acetate and glutamate. Examples of buffers include HEPES-KOH and Tris-acetic acid. Examples of ATP regeneration systems include a combination of phosphoenol pyruvate with pyruvic acid kinase and a combination of creatine phosphate with creatine kinase. Examples of nuclease inhibitors include ribonuclease inhibitor and nuclease inhibitor. Among these, as a specific example of ribonuclease inhibitor, RNase inhibitor derived from human placenta (manufactured by Toyobo) and the like can be used. With regard to tRNA, it is able to be prepared by a method mentioned in Moniter, R., et al., Biochim. Biophys. Acta, 43, 1 (1960), etc. or it which is available in the market may be used. Examples of the reducing agent are dithiothreitol, etc. Examples of the antibacterial agent are sodium azide and ampicillin. With regard to RNA polymerase, that which is suitable as a promoter comprised in a template may be used. To be more specific, SP6 RNA polymerase, T7 RNA, etc. may be used for example. The amount thereof to be added may be appropriately selected to prepare a synthesis reaction solution.

A protein synthesis solution prepared as such is introduced into a selected system or apparatus which has been known per se whereupon protein synthesis is conducted. Examples of the system or the apparatus for the protein synthesis include a batch method (Pratt, J. M., et al., Transcription and Translation, Hames, 179-209, B. D. & Higgins, S. J., eds.), JRL Press, Oxford (1984), a continuous cell-free protein synthesis system where amino acids, energy source, etc. are continuously supplied (Spirin, A. S., et al., Science, 242, 1162-1164 (1988)), a dialysis method (Kigawa, et al., The 21st Japan Molecular Biology Association, WID 6) and a superposition method (Japanese Patent Application No. 2000/259,186). It is also possible to use a method where template RNA, amino acids, energy source, etc. are provided to the synthesis reaction system upon necessity and synthesized and decomposed products are discharged upon necessity (Japanese Patent Laid-Open No. 2000/333,673; hereinafter, that may be referred to as “discontinuous gel filtration method”), a method where the above-mentioned materials for synthesis are developed using the carrier as a mobile phase, synthesis reaction is performed during the development and, as a result, synthesized protein maybe recovered (Japanese Patent Laid-Open No. 2000/316,595), etc. In the protein synthesis reaction used in the present invention, a batch method is considered to be sufficient enough because formation of polyribosome in the initial stage of the translation reaction is an object.

When the protein synthesis is conducted by a batch method, it may be conducted in such a manner, for example, that incubation is carried out by addition of a translation template to the above synthesis reaction solution wherefrom the translation template is removed. When a wheat germ extract is used, incubation is conducted at 10 to 40° C., preferably at 18 to 30° C. or, more preferably, 20 to 26° C. If the reaction time if long enough to generate only polyribosome by a translation template having a high polyribosome forming activity, it is possible to select a nucleic acid sequence having a translation enhancement activity. To be more specific, with regard to the reaction time preferred for selecting the nucleic acid sequence having a translation enhancement activity, a range from 5 minutes to 2 hours is exemplified and, among that, about 30 minutes is exemplified as the best reaction time. Although the reaction time is able to be controlled by stopping the reaction by addition of protein synthesis inhibitory enzyme, the method of the present invention is still able to be conducted even if the reaction is not stopped. With regard to the protein synthesis inhibitory enzyme, any inhibitor may be used so far as it is other than an inhibitor for initiation of the translation reaction. Specific examples include cycloheximide and ribotoxin. With regard to ribotoxin, its specific examples are α-sarcine (Endo, Y., et al., J. Biol. Chem., 258, 2662-2667 (1983)) and ribosome inactivating protein (Endo, Y., et al., J. Biol. Chem., 262, 8128-8130 (1987)). The amount, etc. of these inhibitors that are added may be appropriately selected in the protein synthesis system used and, when cycloheximide is added in a protein synthesis system using a wheat germ extract, it is preferred to be about 0.5 to 10 μM as the final concentration.

When the protein synthesis is conducted by a dialysis method, the synthesis reaction solution to which the translation template is added is used as an inner liquid for dialysis and the protein synthesis is conducted using an apparatus where the outer liquid for dialysis is isolated by a permeable membrane by which mass transfer thereto is possible. To be more specific, a translation template is added to the reaction solution, subjected to incubation for an appropriate time and placed in an appropriate container for dialysis to give an internal liquid for the reaction. With regard to the container for dialysis, a container where a permeable membrane is added to the bottom (such as Dialysis Cup 12,000 manufactured by Daiichi Kagaku) and a tube for dialysis (such as 12,000 manufactured by Sanko Junyaku) maybe exemplified. With regard to a permeable membrane, that having a molecular weight limit of not less than about 1,000 daltons is used and that having a molecular weight limit of about 12,000 daltons is preferred. With regard to an outer liquid for dialysis, the above-mentioned synthesis reaction solution wherefrom the translation template is removed is used. Temperature and time for the reaction may be appropriately selected depending upon the protein synthesis system used.

When the protein synthesis is carried out using a superposition method, it is conducted in such a manner that a synthesis reaction solution to which a translation template is added is placed in an appropriate container and the outer liquid for dialysis mentioned in the above dialysis method is layered on the solution without disarranging the interface. To be more specific, a translation template is added to the above synthesis reaction solution and placed in an appropriate container to give a reaction phase. Examples of the container include a microtiter plate. The outer liquid for dialysis (supplying phase) mentioned in the above dialysis method is layered onto the upper layer of this reaction phase so as not to disarrange the interface and the reaction is conducted. Temperature and time for the reaction are appropriately selected in the protein synthesis system used. It is not always necessary that the interface between both phases is formed in a horizontal form by layering but it is also possible that a mixed solution comprising both phases is centrifuged to form a horizontal plane. When diameter of circular interface between both phases is 7 mm, ratio by volume of the reaction phase to the supplying phase is appropriately from 1:4 to 1:8 and, preferably, it is 1:5. The larger the interface area comprising both phases, the higher the rate of material exchange by diffusion and the higher the efficiency of protein synthesis. Accordingly, the ratio by volume of both phases varies by the interface area of both phases. Synthesis reaction is under the condition of being allowed to stand, and temperature and time for the reaction are appropriately selected in the protein synthesis system used. When an extract of E. coli is used, it is possible to conduct at 30 to 37° C.

When a protein synthesis is conducted using a discontinuous gel filtration method, a synthesis reaction is conducted using a synthesis reaction solution to which a translation template is added and, at the stage where the synthesis reaction stops, template RNA, amino acids, energy source, etc. are supplied and synthesized and decomposed products are discharged to conduct a protein synthesis. To be more specific and for example, a translation template is added to the above-mentioned synthesis reaction solution and placed in an appropriate container to conduct the reaction. Examples of the container include a microplate. Under such a reaction, the synthesis reaction completely stops by the reaction in one hour in the case of a reaction solution comprising, for example, 48% in volume of wheat germ extract. That is able to be confirmed by measuring incorporation of amino acids into protein or by an analysis of polyribosome by a sucrose density gradient centrifugation method (Proc. Natl. Acad. Sci. USA, 97, 559-564 (2000)). The above-mentioned reaction solution where the synthesis reaction has stopped is passed through a gel filtration column pre-equilibrated with a supplying solution having the same composition as the outer liquid for dialysis mentioned in the above dialysis method. When the filtered solution is kept at an appropriate reaction temperature once again, the synthesis reaction is resumed and protein synthesis proceeds for several hours. After that, the reaction and the gel filtration operation as such are repeated. Temperature and time for the reaction are appropriately selected in the protein synthesis system used.

(3) Acquisition of Polyribosome Fractions

The reaction solution for the protein synthesis using a translation template cotaining the candidate polynucleotide of the present invention is fractionated after the reaction to separate a polyribosome fraction. With regard to a method for the fractionation, centrifugal separation method, chromatographic method, filtration method using a filter, etc. are exemplified and a centrifugal separation method is preferably used. With regard to a centrifugal separation method, density gradient centrifugation method, equilibrium density gradient centrifugation method, common fractional centrifugation method, etc. are exemplified and a density gradient centrifugation method is most preferably used.

A density gradient centrifugation method is a method where a sample solution is layered on a pre-prepared density gradient and then centrifuged, and can be conducted by conventional methods well known per se. With regard to an instrument for preparing a density gradient, either commercially available one or a combination of device by a known method may be used so far as a stable density gradient is able to be formed. It is also possible to prepare by layering of solutions having different concentrations. Examples of the solvent which forms the density gradient include sucrose solution, glycerol, heavy water (D₂O) and inorganic salt solution and, among them, sucrose solution is preferably used.

Method for preparation of polyribosome is illustrated in detail by taking that using a sucrose density gradient centrifugation method as an example. In the separation of reaction solution for protein synthesis by a sucrose density gradient centrifugation method, a method mentioned in Proc. Natl. Acad. Sci. USA, 97, 559-564 (2000), etc. maybe used. To be more specific, there is no particular limitation for the concentration gradient of sucrose so far as it is within a range of concentrations by which polyribosome is able to be separated from the above reaction solution for protein synthesis and, usually, a concentration gradient where its lower limit is within a range of 5 to 30% and its upper limit is within a range from 30% to a saturation concentration is used. Among the above, the concentration gradient between the lower limit of 10% and the upper limit of 60% is most preferably used. With regard to a buffer which dissolves sucrose, anything may be used so far as it is able to keep a complex of polyribosome with translation template in as table manner and, to be more specific, that which comprises Tris-HCl, potassium chloride, magnesium chloride, cycloheximide, etc. may be exemplified.

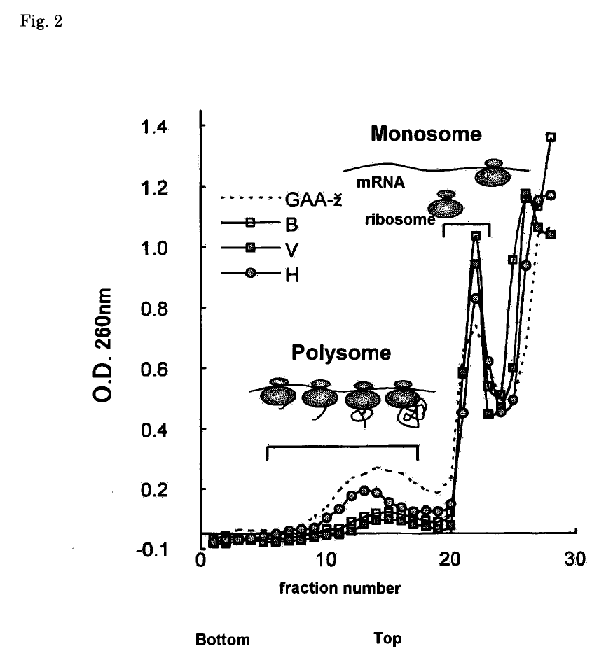

A density gradient by the sucrose solution as such is prepared on an appropriate centrifugal tube and a synthetic solution of protein after completion of the reaction is layered after, if necessary, diluting with an appropriate buffer. With regard to the appropriate buffer, similar one used for dissolving of sucrose is preferably used. With regard to the degree of dilution, there is no particular limitation so far as no coprecipitation takes place and, preferably, dilution to an extent of about 1- to 100-fold is conducted. The diluted reaction solution for protein synthesis is able to be layered in an amount of about 1/100 to 100-fold to the sucrose solution and, preferably, about 1/50 fold is layered. That is centrifuged to such extent that polyribosome is separated. To be more specific, condition for the centrifugation is, for example, 80,000 to 400,000×g at 4° C. for 30 minutes to 3 hours. After completion of the centrifugation, that is fractionated into an appropriate amount each and nucleic acid amount in each fraction is measured, etc. whereupon a fraction in which polyribosome is contained is identified. To be more specific, when a density gradient centrifugation is conducted by, for example, 5 ml of sucrose solution and 100 μl of protein synthesis reaction solution, each 100 to 200 μl is taken as one fraction and OD₂₆₀ for each fraction is measured. When a protein synthesis system derived from eukaryotes, for example, is used, there is a peak showing 80S ribosome in the measured value and fractions such as those where the fraction in which the measured value shows a peak is the center existing in the side of more molecular weight than the above are prepared as a polyribosome fraction. Examples of such a polyribosome fraction include fractions 13 to 23 of Example 1 (sucrose concentration: 32.5 to 45%) or fractions 13 to 21 of Example 2 (sucrose concentration: 35 to 45%) which will be mentioned later. When the fractions are shown as a graph in terms of measured values of OD₂₆₀, they are the ranges shown by enlarged graphs of FIG. 1 and FIG. 2.

(4) Screening for Nucleic Acid Sequence Having an Activity of Regulating the Translation Efficiency and Acquisition of Polynucleotide Containing the Sequence

RNA is recovered from the polyribosome fraction prepared as such and the resulting RNA is subjected to a reverse transcription reaction to prepare cDNA. When a sequence of a candidate polynucleotide part contained in the cDNA is analyzed, it is possible to select a nucleic acid sequence having an activity of regulating the translation efficiency. The above cDNA comprises a polynucleotide containing a nucleic acid sequence having an activity of regulating the translation efficiency and, when the sequence part is amplified by, for example, means of a PCR, it is possible to prepare a polynucleotide comprising a nucleic acid sequence having an activity of regulating the translation efficiency.

With regard to a method for the preparation of RNA bonding to polyribosome from the polyribosome fraction, it is possible to use a method well known per se and, to be more specific, an acid guanidium thiocyanate-phenol-chloroform (AGPC) method (Chomczynski, P. et al., Anal. Biochem., 162, 156-159 (1987)) is preferably used. There is a possibility that the solution containing RNA prepared here comprises DNA which is introduced into the protein synthesis system as a translation template and, therefore, it is preferred to subject to a treatment with a DNA-decomposing enzyme such as DNase I.

The resulting RNA solution is purified by a conventional method such as extraction with phenol/chloroform or precipitation with ethanol and able to be subjected to a reverse transcription reaction. With regard to the reverse transcription reaction, it is possible to use a known method which has been commonly used and, in view of production efficiency, etc. of cDNA, it is preferred to use an AMV reverse transcriptase. It is also possible to use a commercially available kit such as an RNA LA PCR Kit (AMV) ver. 1.1 (manufactured by Takara).

The cDNA per se prepared by the reverse transcription reaction comprises a polynucleotide which has an activity of regulating the translation efficiency and it is also able to be cloned or amplified. When it is cloned, the above-prepared cDNA may be inserted into an appropriate vector and cloned. When a common sequence is added in the preparation of a translation template containing a candidate polynucleotide in (1), it is also possible that amplification is conducted by PCR using an antisense primer having a homology to the common sequence and to a sequence of 5′-terminal of a coding polynucleotide followed by inserting into an appropriate vector to be cloned. With regard to the polynucleotide cloned as such, a translation template is prepared in a similar manner using it as a candidate polynucleotide of (1) and protein synthesis is conducted using the translation template whereupon its activity of regulating translation efficiency is able to be confirmed. With regard to a quantitative determination method of the synthesized amount of the protein, its specific examples used include the measurement of incorporation of amino acids into protein, the separation by an SDS-polyacrylamide electrophoresis followed by staining with Coomassie Brilliant Blue (CBB) and an autoradiographic method (Endo, Y., et al., J. Biotech, 25, 221-230 (1992); Proc. Natl. Acad. Sci. USA, 97, 559-564 (2000)). When a substance coding for fluorescent protein such as luciferase and GFP is used as a translation template for the present invention, a method where fluorescence intensity generated from the protein is measured is preferably used. When luciferase is used, its sequence length of gene is longer than GFP. Therefore, entry of more ribosomes than GFP is presumed and bigger ribosome is formed whereby an effect that ribosome is able to be easily fractionated is expected as well. In addition, when a sequence of the cDNA is analyzed by a commonly used method, it is possible to identify the nucleic acid sequence having the activity of regulating the translation efficiency.

(5) Screening of a Nucleic Acid Sequence with Still Higher Activity of Regulating the Translation Efficiency and Method for Acquisition of Polynucleotide Comprising the Sequence.

When a translation template is prepared by the same manner using the cDNA prepared in the method mentioned in the above (4) is used as a candidate polynucleotide for the above (1) and the methods mentioned in the above (1) to (4) are repeated, it is possible to acquire a polynucleotide with still higher activity of regulating the translation efficiency and to identify its sequence. In addition, when mutation is introduced by a commonly used method known per se into the cDNA prepared in the above (4) and the methods mentioned in the above (1) to (4) are repeated using the mutant, it is also possible to acquire a polynucleotide with still higher activity of regulating the translation efficiency and to identify its sequence. Specific examples for introducing the mutation into a sequence include an error-prone PCR method and a point mutagenesis method.

Among the polynucleotide having an activity of regulating the translation efficiency prepared as such, examples of those having a translation enhancement activity include that comprising a sequence shown in SEQ ID NO: 8 in the Sequence Listing. The polynucleotide which is screened and prepared as such comprises an artificially random sequence and does not contain a sequence existing in nature. The polypeptide of the present invention also comprises an artificial random nucleic acid sequence having a length of 30 to 200 mer and methods for screening and acquisition thereof are not limited to the above-mentioned ones so far as the product has an activity of regulating the translation efficiency.

An embodiment of the polynucleotide according to the present invention comprises a sequence mentioned in SEQ ID NO: 8 or a complementary sequence of the above sequence and is a polynucleotide having a translation enhancement activity.

A sequence having a homology to the sequence mentioned in SEQ ID NO: 8 and a polynucleotide having a complementary sequence to the sequence and having a translation enhancement activity are also within a scope of the present invention. It is desirable that the sequence homology is usually not less than about 50%, preferably not less than about 70%, more preferably not less than about 80% and, still more preferably, not less than about 90% of the total sequence.

The polynucleotide according to the present invention includes a polynucleotide which comprises a sequence where there is/are mutation(s) such as deletion, substitution, addition or insertion of not less than one such as 1 to 50, preferably 1 to 30, more preferably 1 to 20, still more preferably 1 to 10 or, particularly preferably, 1 to several nucleotide(s) in the sequence mentioned in SEQ ID NO: 8 and has a translation enhancement activity.

There is no particular limitation for degree and positions of the mutation so far as the polynucleotide having the mutation has a biological function which is in the same quality as the above-mentioned polynucleotide has. A polynucleotide having such mutation maybe that which is present in nature or that which is prepared by induction of mutation on the basis of gene derived from nature.

With regard to means for induction of mutation, it is known per se and, for example, site-specific mutation induction method, gene homology recombination method, primer elongation method and PCR may be used either solely or jointly by an appropriate combination thereof. For example, that may be carried out according to a method mentioned in already-available books (“Molecular Cloning, a Laboratory Manual; Second Edition” edited by Sambrook, et al., 1989, Cold Spring Harbor Laboratory; and “Labo Manual—Genetic Engineering” edited by Masami Muramatsu, 1988, Maruzen) or by modifying those methods. It is also possible to utilize a technique of Ulmer (Ulmer, K. M., Science, 219, p.666-671 (1983)).

The polynucleotide according to the present invention includes a polynucleotide which hybridizes to the above-mentioned polynucleotide under a stringent condition. Conditions for the hybridization may be followed, for example, a method mentioned in already-available books such as Proceedings of The National Academy of Sciences of The United States of America, 74, p. 5463-5467 (1977).

(6) Protein Synthesis by a Translation Template Containing a Polynucleotide Having a Translation Enhancement Activity

When the polynucleotide of the present invention having a translation enhancement activity is bonded in such a manner of being sandwiched between a promoter sequence and a coding polynucleotide which codes for an aimed polypeptide, a translation template is able to be prepared. It is preferred that the coding polynucleotide comprises not only a coding region for the above-mentioned polynucleotide but also a 3′-untranslated region including a transcription termination region thereof, etc. With regard to the 3′-untranslated region, about 0.1 to 3.0 k base(s) being in a downstream side from stop codon is preferably used. With regard to a promoter, that which is specific to RNA polymerase being used for transcription thereafter may be used. Specific examples include SP6 promoter and T7 promoter

With regard to a method for bonding of promoter and coding polynucleotide with the polynucleotide of the present invention having a translation enhancement activity, a method mentioned in the above (1), an overlap PCR method, etc. may be used. The translation template prepared as such is subjected to a protein synthesis system in the same manner as that mentioned in the above (2) whereupon an aimed polypeptide is able to be synthesized. The polypeptide prepared as such is able to be confirmed by a method which is known per se. To be more specific, it is possible to use the measurement of incorporation of amino acids into protein, the separation by SDS-polyacrylamide electrophoresis followed by staining with Coomassie Brilliant Blue (CBB), an autoradiographic method (Endo, Y., et al., J. Biotech., 25, 221-230 (1992); Proc. Natl. Acad. Sci. USA, 97, 559-564 (2000) and the like.

Since the reaction solution prepared as such comprises the aimed protein in a high concentration, the aimed polypeptide is able to be prepared by subjecting the reaction solution to known separation and purification methods known per se such as dialysis, ion exchange chromatography, affinity chromatography or gel filtration.

(7) Vector Containing the Polynucleotide Having a Translation Enhancement Activity

When the polynucleotide having a translation enhancement activity according to the present invention is inserted into an appropriate vector, it is possible to construct a vector for the preparation of translation template for protein synthesis. Examples of the vector used include appropriate cloning vector, T7 promoter and vector for protein synthesis containing SP6 promoter or transcription termination region.

### EXAMPLES

The present invention will now be illustrated in detail by way of the following Examples although the scope of the present invention is not limited by those Examples.

### Example 1

### Selection of Nucleic Acid Sequence Having a Translation Enhancement Activity

(1) Preparation of RNA Containing a Candidate Polynucleotide (Random Sequence)

A PCR using a plasmid in which luciferase gene DNA (pSP-luc⁺: manufactured by Promega, catalog number: E1781) was inserted as a translation template was carried out using a sense primer (SEQ ID NO: 1) comprising a sequence having a randomized site of 30 to 200 mer, an A for endowing a Kozak sequence to 3′ side thereof and a sequence of 5′-terminal of DNA of luciferase gene at 3′ side thereof and being connected with a common sequence 12 nts at 5′ side of the randomized-site and also SP6 promoter at 5′ side thereof and an antisense primer (SEQ ID NO: 2) containing a sequence of downstream side 3′ to an extent of 1652 bases from stop codon of luciferase gene DNA. The resulting DNA fragment of about 3,400 bp was purified by ethanol precipitation and was used as a template for conducting a transcription using an SP6 RNA polymerase (manufactured by TAKARA) and the resulting RNA was extracted with phenol/chloroform and precipitated with ethanol and purified by a Nick Column (manufactured by Amersham Pharmacia Biotech). That was used as a translation template for the following experiments.

(2) Preparation of Solution Containing a Wheat Germ Extract

Seeds of wheat (chihoku, a product of Hokkaido) were added to a mill (Rotor Speed Mill Pulverisette, type 14; manufactured by Fritsch) at the rate of 100 g per minute and the seeds were gently ground at 8,000 rpm. After a fraction (mesh size: 0.7 to 1.00 mm) containing germinatable germs was recovered using a sieve, a floated fraction containing germinatable germs was recovered by means of floating using a mixed liquid of carbon tetrachloride with cyclohexane (ratio by volume of carbon tetrachloride to cyclohexane=2.4:1), then organic solvents were removed by drying at room temperature and impurities such as seed coat contaminated therein were eliminated by air-blowing at room temperature to give a crude germ fraction. Wheat germ was discriminated from the crude germ fraction by naked eye and selection was conducted using a bamboo skewer.

The resulting wheat germ fraction was suspended in distilled water of 4° C. and washed with an ultrasonic washing machine until the washing did not show any turbidity. After that, it was suspended in a 0.5% by volume solution of Nonidet P40 (manufactured by Nakarai Techtonics) and washed with an ultrasonic washing machine until the washing did not show any turbidity to obtain wheat germs.

Preparation of a solution containing the wheat germs was conducted according to a common method (Erickson, A. H., et al., (1996), Meth. In Enzymol., 96, 38-50). The following operation was conducted at 4° C. Firstly, the wheat germs frozen with liquid nitrogen were finely disintegrated in a mortar. To 1 g of the resulting powder was added 1 ml of an extracting solvent of Patterson, et al. which was partially modified (containing 80 mM of HEPES-KOH (pH 7.6), 200 mM of potassium acetate, 2 mM of magnesium acetate, 4 mM of calcium chloride, each 0.6 mM of 20 kinds of L-amino acids and 8 mM of dithiothreitol in terms of final concentrations) and the mixture was carefully stirred so as not to generate foams. The supernatant liquid obtained by centrifugation of 30,000×g for 15 minutes was recovered as germ extract and was subjected to a gel filtration using a Sephadex G-25 column (manufactured by Amersham Pharmacia Biotech) which was previously equilibrated with a solution (containing 40 mM of HEPES-KOH (pH 7.6), 100 mM of potassium acetate, 5 mM of magnesium acetate, each 0.3 mM of 20 kinds of L-amino acids and 4 mM of dithiothreitol in terms of final concentrations). Concentration of the solution containing the wheat germ extract obtained as such was prepared in such a manner that optical density at 260 nm (O.D.) (A 260) was 170 to 250 (A 260/A 280=1.5).

(3) Protein Synthesis by a Cell-Free Protein Synthesis System (Batch Method) Using Wheat Germ Extract

A reaction solution (25 μl) for protein synthesis containing 5.8 μl of the solution containing the wheat germ extract prepared in the above (2) was prepared (29 mM of HEPES-KOH (pH 7.8), 95 mM of potassium acetate, 2.7 mM of magnesium acetate, 0.4 mM of spermidine (manufactured by Nacarai Techtonics), each 0.23 mM of 20 kinds of L-amino acids, 2.9 mM of dithiothreitol, 1.2 mM of ATP (manufactured by Wako Pure Chemical), 0.25 mM of GTP (manufactured by Wako Pure Chemical), 15 mM of creatine phosphate (manufactured by Wako Pure Chemical), 0.9 U/μl of RNase inhibitor (manufactured by Takara), 50 ng/μl of tRNA (Moniter, R., et al., Biochim. Biophys. Acta, 43, 1-(1960)) and 0.46 μg/l of creatine kinase (manufactured by Roche) in terms of final concentrations) mRNA (8 μg) containing random sequence prepared in the above (1) was added to the reaction solution and incubation was conducted at 26° C. for 30 minutes. After 30 minutes, cycloheximide (manufactured by Wako Pure Chemical) was added thereto so as to make its final concentration 1.5 μM whereupon protein synthesis was stopped.

(4) Preparation of Sucrose Density Gradient

Each 2.5 ml of a 10% sucrose solution (containing 25 mM of Tris-HCl (pH 7.6), 50 mM of potassium chloride, 5 mM of magnesium chloride, 10% of sucrose (manufactured by Nakarai Techtonics) and 0.75 μM of cycloheximide (manufactured by Wako Pure Chemical) in terms of final concentrations) and a 60% sucrose solution (containing 25 mM of Tris-HCl (pH 7.6), 50 mM of potassium chloride, 5 mM of magnesium chloride, 60% of sucrose (manufactured by Nakarai Techtonics) and 0.75 μM of cycloheximide (manufactured by Wako Pure Chemical) in terms of final concentrations) were placed in a centrifugal tube where the 60% sucrose solution was in lower layer while the 10% sucrose solution was in upper layer. After that, density gradient was prepared using a gradiater (Biocomp-Gradent Master manufactured by Towa Kagaku) with the following settings (First run, speed: 25 rpm, angle: 55 deg, time: 1 min 50 sec; second run, speed: 25 rpm, angle 83.5 deg, time: 1 min 25 sec). The density gradient solution prepared as such was allowed to stand at 4° C. for 3 hours.

(5) Separation of Polyribosome Fraction by a Sucrose Density Gradient Centrifugal Separation and Extraction of RNA

A diluted solution (75 μl) (containing 25 mM of Tris-HCl (pH 7.6), 50 mM of potassium chloride and 5 mM of magnesium chloride (manufactured by Nakarai Techtonics) in terms of final concentrations) was added to the reaction solution after stopping of protein synthesis in the above (3), placed on the sucrose density gradient solution prepared in the above (4) and centrifuged at 40,000 rpm for 1 hour at 4° C. (centrifugal machine: CP650β; rotor: P55ST2; manufactured by Hitachi). After that, each 100 to 120 μl of fraction was taken out and O. D. 260 nm for each fraction was measured. The result is shown in FIG. 2. Fractions of 13 to 23 (sucrose concentrations: 32.5 to 45%) where it is likely that protein synthesis proceeds smoothly and polyribosome was found was subjected to an extraction of RNA by an AGPC method (Chomczynski, P., et al., Anal. Biochem., 162, 156-159 (1987)). DNase I (25 U) (manufactured by TAKARA) was added to the whole amount of the extract, incubation was conducted at 37° C. for 15 minutes to decompose the remaining DNA and, after that, purification was conducted by phenol/chloroform extraction and ethanol precipitation.

(6) Preparation of cDNA and Amplification

A reverse transcription reaction solution (containing 5 mM of magnesium chloride, 1× RNA PCR buffer, 1 mM of DNTP mixture, 1.0 μM of antisense primer (SEQ ID NO: 2), 1 U/μl of RNase inhibitor and 0.25 U/μl of reverse transcriptase in terms of final concentrations) was prepared using an RNA LA PCR Kit (AMV) ver. 1.1 (manufactured by TAKARA) and the whole amount of the RNA extract of the above (5) was added thereto as a template to conduct a reverse transcription reaction whereupon cDNA was prepared. In order to amplify the cDNA, the reverse transcription product (1 μl) was used as a template and a PCR was conducted using a sense primer (SEQ ID NO: 3) containing common sequence, 3′-terminal GAA sequence of SP6 promoter at 5′-side thereof, where appropriate five-sequence was further bonded to 5′-side thereof and an antisense primer (SEQ ID NO: 4) containing a sequence of 20th base from A of start codon of luciferase gene DNA whereupon about 150 bp of DNA fragments were prepared. To this were added 5 U of exonuclease I (manufactured by USB), incubation was conducted at 37° C. for 30 minutes and then incubation was conducted at 80° C. for 30 minutes to inactivate Exonuclease I. After that, the whole amount was recovered from agarose gel using a GFX™ PCR DNA and Gel Band Purification Kit (manufactured by Amersham Pharmacia Biotech)

### Example 2

### Screening of Nucleic Acid Sequence Having Translation Enhancement Activity and Acquisition of Polynucleotide Comprising the Sequence

(1) Preparation of RNA Containing a Candidate Polynucleotide (Random Sequence) in the Second Run

A plasmid into which luciferase gene DNA was inserted was used as a template and a PCR was carried out using a sense primer (SEQ ID NO: 5) having a complementary sequence to an antisense primer mentioned in SEQ ID NO: 4 containing a sequence which was 20th base from A of start codon of luciferase gene DNA and an antisense primer (SEQ ID NO: 6) containing a sequence which was downstream to an extent of 2 bases from 3′ of the sequence shown in SEQ ID NO: 2 whereupon DNA fragments of about 3,200 bp partially containing luciferase gene DNA were prepared. The PCR product (1 μl) and a DNA fragment which was in an amount of 1/50 of the DNA fragment of about 150 bp recovered in Example 1(6) were used as templates and a PCR was conducted again using a sense primer (SEQ ID NO: 1) and an antisense primer (SEQ ID NO: 2) whereupon DNA fragments of about 3,400 bp were prepared. A three-fourth amount thereof was used as a template and a transcription was conducted using SP6 RNA polymerase (manufactured by TAKARA) and the resulting RNA was extracted with phenol/chloroform, precipitated with ethanol and purified by a Nick Column (manufactured by Amersham Pharmacia Biotech). That was used as a translation template for the following experiments.

(2) Protein Synthesis by a Wheat Germ Cell-Free Protein Synthesis System (Batch Method) in the Second Run

A reaction solution (25 μl) for the synthesis of protein containing 5.8 μl of a solution containing a wheat germ extract prepared in Example 1 (2) was prepared (under the same condition as in Example 1 (3)). To this reaction solution was added 8 μg of mRNA containing the candidate polynucleotide prepared in Example 1 (7) and incubation was conducted at 26° C. for 30 minutes. After 30 minutes, cycloheximide (manufactured by Wako Pure Chemical) was added so as to make its final concentration 1.5 μM whereupon the protein synthesis was stopped.

(3) Separation of Polyribosome Fraction by a Sucrose Density Gradient Centrifugal Separation and Extraction of RNA in the Second Run

A diluted solution (under the same condition as in Example 1 (5)) was added to the reaction solution after stopping the protein synthesis of the above (2) and the mixture was placed on the sucrose density gradient solution prepared in Example 1 (4) and centrifuged at 40,000 rpm for 1 hour at 4° C. (centrifuge: CP650β; rotor: P55ST2; manufactured by HITACHI). After that, each 100 to 120 μl of fraction was taken out and optical density (O. D.) of each fraction at 260 nm was measured. The result is shown in FIG. 2. The fractions 13 to 21 (sucrose concentration: 35 to 45%) where polyribosome was noted in which the protein synthesis was thought to be smoothly proceeded were subjected to extraction of RNA by an AGPC method (Chomczynski, P ., et al., Anal. Biochem., 162, 156-159 (1987)). To the whole extract was added 25 U of DNase I (manufactured by TAKARA), incubation was conducted at 37° C. for 15 minutes to decompose the remaining DNA and, after that, purification was conducted by phenol/chloroform extraction and ethanol precipitation.

(4) Preparation of cDNA and Amplification

A reverse transcription reaction was conducted under the same condition as in Example 1 (6) to prepare cDNA. In order to further amplify the cDNA, a polymerase chain reaction (PCR) was conducted using a primer having the sequences mentioned in SEQ ID NO: 3 and NO: 7 and using 1 μl of the reverse transcription product as a template to give DNA fragments of about 150 bp.

(5) TA Cloning and Sequencing

A reaction solution (containing 1× Rapid Ligation Buffer and ng/μl pGEM-T Easy Vector in terms of the final concentrations) was prepared using pGEM-T Easy Kit (manufactured by Promega), the DNA fragments of the above (4) were added thereto and incubation was conducted at 14° C. for 4 hours whereupon DNA fragments were integrated into pGEM-T Easy Vector. After that, a transformation was conducted to E. coli JM 109 (manufactured by Takara) using the whole amount, plasmid was extracted from the resulting colonies and sequencing was conducted for the inserted sequence in the plasmid. As a result, one kind of novel sequence (SEQ ID NO: 8) was confirmed in a randomized site.

### Example 3

### Investigation of Translation Enhancement Activity of the Novel Sequence

(1) Preparation of DNA Fragments Containing Novel Sequence

With regard to mRNA which is to be a translation template, it was used after such a manner that transcription was conducted by an SP6 RNA polymerase (manufactured by Promega) using a cyclic plasmid where Ω sequence part was substituted with a sequence mentioned in SEQ ID NO: 8 as a template on the basis of pEU-GFP vector (Sawasaki, T., et al., PNAS, 99 (23), 14652-7 (2002) ) into which GFP gene DNA (Chiu, W., et al., Curr. Biol., 6, 325-330 (1996)) was inserted and the resulting DNA was extracted with phenol/chloroform and precipitated with ethanol and purified by a Nick Column (Amersham Pharmacia Biotech). Further, as a sequence having a translation enhancement activity for the control, DNA fragments where an omega (Ω) sequence of tobacco mosaic virus (TMV) was contained in 5′-untranslated region and 3′-untranslated region was about 1,600 nts were transcribed, purified and used as a control.

(2) Protein Synthesis by a Wheat Germ Cell-Free Protein Synthesis System (Batch Method)

A reaction solution (25 μl ) containing 5.8 μl of a solution containing wheat germ extract prepared in Example 1 (2) (containing 29 mM of HEPES-KOH (pH 7.8), 95 mM of potassium acetate, 2.7 mM of magnesium acetate, 0.4 mM of spermidine (manufactured by Nakarai Techtonics), each 0.23 mM of 20 kinds of L-amino acids, 2.9 mM of dithiothreitol, 1.2 mM of ATP (manufactured by Wako Pure Chemical), 0.25 mM of GTP (manufactured by Wako Pure Chemical), 15 mM of creatine phosphate (manufactured by Wako Pure Chemical), 0.9 U/μl of RNase inhibitor (manufactured by TAKARA), 50 ng/μl of tRNA (Moniter, R., et al., Biochim. Biophys. Acta, 43, 1-(1960)) and 0.46 μg/l of creatine kinase (manufactured by Roche) in terms of the final concentrations) for protein synthesis was prepared. To this solution was added 8 μg/ml of a template mRNA followed by incubating at 26° C.

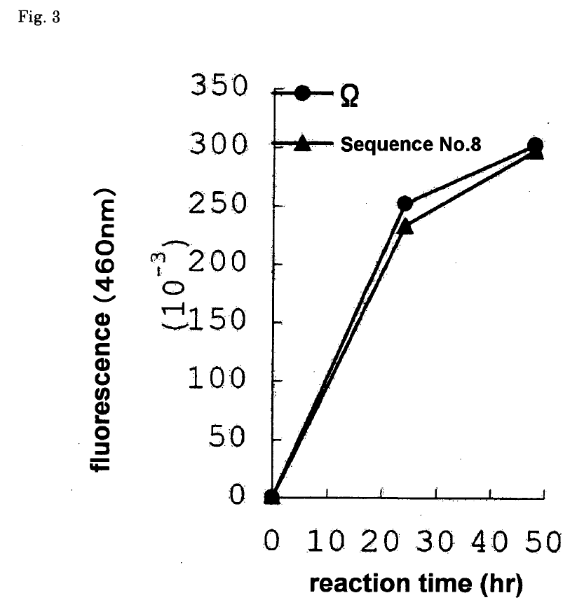

After initiation of the reaction, each 1 μl of the reaction solution until 48 hours was diluted to 100-fold and subjected to measurement of fluorescence at 460 nm using a luminometer (TD-360 manufactured by Turner Designs). The result is shown in FIG. 3.

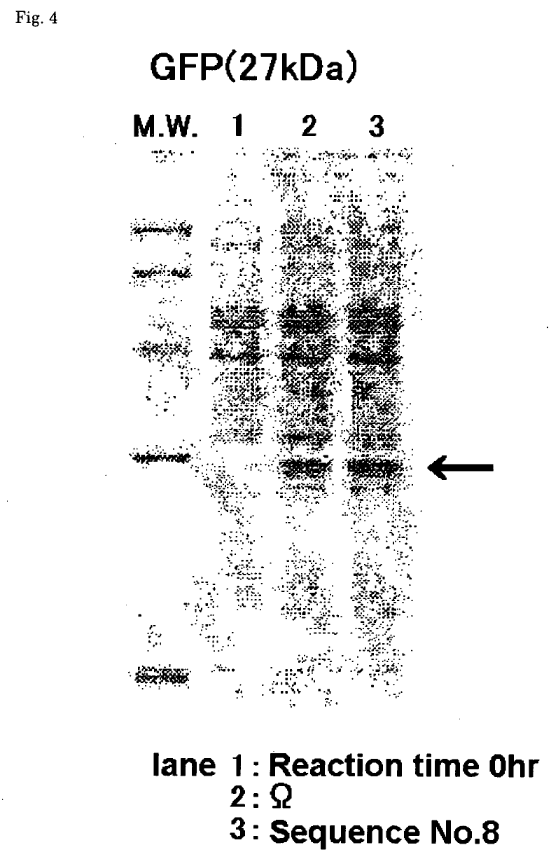

From the reaction solution of each of the above-mentioned synthesis systems, 1 μl was collected after 48 hours, separated using a 12.5% SDS-polyacrylamide electrophoresis (SDS-PAGE) and analyzed by staining with Coomassie Brilliant Blue (CBB) The result is shown in FIG. 4. The sequence mentioned in SEQ ID NO: 8 showed the same translation template activity as in the case of RNA containing Ω sequence.

This application claims priority from Japanese Patent Application No. 2004/227866, which is incorporated herein by reference.

1. A polynucleotide having a translation enhancement activity containing the following sequence:
1) nucleic acid comprising a sequence represented by SEQ ID NO: 8 of the Sequence Listing and/or a complementary chain thereof,
2) nucleic acid coding for a sequence comprising a sequence where one to several nucleotide(s) in a sequence represented by SEQ ID NO: 8 of the Sequence Listing is/are substituted, deleted, inserted or added and/or a complementary chain thereof,
3) nucleic acid having at least 80% of homology to a sequence represented by SEQ ID NO: 8 of the Sequence Listing and/or a complementary chain thereof.

2. A polynucleotide having a translation enhancement activity containing a sequence which is hybridized to the sequence mentioned in claim 1 under a stringent condition or a polynucleotide comprising a complementary sequence thereof.

3. The polynucleotide according to claim 1, wherein the activity of regulating the translation efficiency is identical with or higher than the activity of a 5′-untranslated leader sequence of RNA virus.

4. A translation template containing the polynucleotide according to claim 1.

5. A method for the protein synthesis wherein the translation template mentioned in claim 4 is used.

6. A protein which is prepared by the method for the protein synthesis mentioned in claim 5.

7. A vector containing the polynucleotide according to claim 1.
